SEQUENCE LISTING (6 sequences)

SEQ ID NO: 1 - DNA, 1774 bp, Cucumis melo
  source 1..1774: mol_type = genomic DNA
aaataaaaatatttacagtggatagaatggaggaattatataagaaacgaggataccaatttgcaaccattcttttattgatgaaggaaagaaaatgaaaatacaaataaagagtccaaattggcatatttggtggtaaggtaaggtaaggtgcattcacaccaaccattcttttccaattttcctaatcttttacatttttctctactatcctaaattagcttcaatttaattctttttcttttcttttttaatttggttaaccatcttttttaaactaccttatgcactatttaacttttaaaaaaattgttatgaatagaaaaatatatatatatattatttataaaatataacaaaaacttaaatagaaaatagtttccatattttgctactttggaaaatgtttcttaattttaataatatgtgattttttttattgatttatgtgtgaagtaaaaaatgaaaaagaaaatataggaatgtatcagtttatgtgtttctaaaacttagattaatttatgcttctatttttttttttttttatttactaatctcatgccaaattagttaaatatttacaataactatatcatgccgtgatttagtaaatggtgtaaagtgaaaaaaaaaattaaataaaattctaattctgctctctatgaatttttaatacaattttttatagaaaaatttaagtccactagaaaagaaggtatataggtagatgtaggaaaatctctcactctacgcatcatttcataactcaaaaaaaaaaaaagaatgaataaataaatagaatactacatcaaataaactgtggttttgttaatattaaccataaaaatgtgctatattcattttaagttacacattcattgatagaatatgttgattaaaaattcaacgaaagacagctaaattgcattaatttcagagaatatgttatggatgttgtgatcatattcaagttccaatgggaagttgtcaatgatgaattggatccgttagtgtgttaaaattttattaataccacagataaacacattataattctggaagaaagctagcttgattcaatatataattaaagactttttttttctctctcttttttttttttttttttttttaattttggagatttgatttttcgttcacattatatatgctcattctatgaatatggtcacaaaacatttaaagtcaatttatttctttatttatcacaaacataattttcctttttgtttccgtacaaaattaaatcaactctctcccaattttactaatatcacacgtatttcttactactataatttctctatctttatacactaaccttaccatatacttttttttttatcttcaagatatttcacctcccggcctattatcacttcctcgatatgacatacagtcataatccgtaagtgtgcttctttttatcttaggatcacgttggtttttggttttttctatgacagtcgtttaatacagttgctccaactcatttatcgtgggacacactaatataatcaaatatctaacttccacacagccaaaacaagatgttttatcaacacaagaaaggtgatactttcaaacgttcaaacatcattgttgttaatgccatgtaacacactactacctccggctttccgtttacactttcttttatttatttatctatttatttaaaagttttgaagaaacggcttttcaatgtatggtaacatttttcataaattcaaattaaattattgtatttgaa

SEQ ID NO: 2 - DNA, 1774 bp, Cucumis melo
  source 1..1774: mol_type = genomic DNA
aaataaaaatatttacagtggatagaatggaggaattatataagaaacgaggataccaatttgcaaccattcttttattgatgaaggaaagaaaatgaaaatacaaataaagagtccaaattggcatatttggtggtaaggtaaggtaaggtgcattcacaccaaccattcttttccaattttcctaatcttttacatttttctctactatcctaaattagcttcaatttaattctttttcttttcttttttaatttggttaaccatcttttttaaactaccttatgcactatttaacttttaaaaaaattgttatgaatagaaaaatatatatatatattatttataaaatataacaaaaacttaaatagaaaatagtttccatattttgctactttggaaaatgtttcttaattttaataatatgtgattttttttattgatttatgtgtgaagtaaaaaatgaaaaagaaaatataggaatgtatcagtttatgtgtttctaaaacttagattaatttatgcttctatttttttttttttttatttactaatctcatgccaaattagttaaatatttacaataactatatcatgccgtgatttagtaaatggtgtaaagtgaaaaaaaaaattaaataaaattctaattctgctctctatgaatttttaatacaattttttatagaaaaatttaagtccactagaaaagaaggtatataggtagatgtaggaaaatctctcactctacgcatcatttcataactcaaaaaaaaaaaaagaatgaataaataaatagaatactacatcaaataaactgtggttttgttaatattaaccataaaaatgtgctatattcattttaagttacacattcattgatagaatatgttgattaaaaattcaacgaaagacagctaaattgcattaatttcagagaatatgttatggatgttgtgatcatattcaagttccaatgggaagttgtcaatgatgaattggatccgttagtgtgttaaaattttattaataccacagataaacacattataattctggaagaaagctagcttgattcaatatataattaaagactttttttttctctctcttttttttttttttttttttttaattttggagatttgatttttcgttcacattatatatgctcattctatgaatatggtcacaaaacatttaaagtcaatttatttctttatttatcacaaacataattttcctttttgtttccgtacaaaattaaatcaactctctcccaattttactaatatcacacgtatttcttactactataatttctctatctttatacactaaccttaccatatacttttttttttatcttcaagatatttcacctcccggcctattatcacttcctcgatatgacatacagtcataatccgtaagtgtgcttctttttatcttaggatcacgttggtttttggttttttctatgacagtcgtttaatacagttgctccaactcatttatcgtgggacacactaatataatcaaatatctaacttccacacagccaaaacaaaatgttttatcaacacaagaaaggtgatactttcaaacgttcaaacatcattgttgttaatgccatgtaacacactactacctccggctttccgtttacactttcttttatttatttatctatttatttaaaagttttgaagaaacggcttttcaatgtatggtaacatttttcataaattcaaattaaattattgtatttgaa

SEQ ID NO: 3 - DNA, 200 bp, Cucumis melo
  source 1..200: mol_type = genomic DNA
gtgtaaacggaaagccggaggyagtagtgtgttacatggcattaacaacaatgatgtttgaacgtttgaaagtatmacctttcttgtgttgataaaacatctttgttttggctgtgtggaagttagatatttgattatattagtgtgtcccacgataaatgagttggagcaactgtattaaacgactgtcatagaaaaaa

SEQ ID NO: 4 - DNA, 30 bp, synthetic construct
  source 1..30: mol_type = other DNA
gtatmacctttcttgtgttgataaaacatc

SEQ ID NO: 5 - DNA, 30 bp, synthetic construct
  source 1..30: mol_type = other DNA
gtatmacctttcttgtgttgataaaacatt

SEQ ID NO: 6 - DNA, 29 bp, synthetic construct
  source 1..29: mol_type = other DNA
gtcgtttaatacagttgctccaactcatt

(19) United States
(12) Patent Application Publication
(10) Pub. No.: US 2026/0033446 A1
(43) Pub. Date: Feb. 5, 2026

(54) MELONS WITH IMPROVED SHELF LIFE

(71) Applicant: RIJK ZWAAN ZAADTEELT EN ZAADHANDEL B.V., De Lier (NL)

(72) Inventors: Magdalena Barbara LASTDRAGER, De Lier (NL); Paul DE GRAAG, De Lier (NL)

(21) Appl. No.: 19/293,561

(22) Filed: Aug. 7, 2025

Related U.S. Application Data
(63) Continuation-in-part of application No. PCT/EP2024/053192, filed on Feb. 8, 2024.

(30) Foreign Application Priority Data
Feb. 9, 2023 (WO) PCT/EP2023/053254

(51) Int. Cl. A01H 1/04; C12Q 1/6895

(52) U.S. Cl. CPC A01H 1/045; C12Q 1/6895; C12Q 2600/13; C12Q 2600/156

(57) ABSTRACT

The present invention relates to Cucumis melo plant having a modified lncRNA gene according to SEQ ID NO: 1, or according to a sequence having at least 50% sequence identity to SEQ ID NO: 1, having a modification, wherein the modification comprises an insertion, a deletion or a substitution of at least one nucleotide. The modified lncRNA gene has a changed function, a reduced function, or is non-functional. The modification is suitably in the second exon and can be a G1571A mutation at position 1571 in SEQ ID NO: 1. Presence of the modified lncRNA gene confers improved shelf life of the fruits, when compared to fruits of an isogenic melon plant lacking the modified lncRNA gene. The improved shelf life of the fruits is characterized by a prolonged firmness of the fruit flesh, and/or by a delay of the ethylene peak during ripening of the fruits.

### RELATED APPLICATIONS AND INCORPORATION BY REFERENCE

This application is a continuation-in-part application of international patent application Serial No. PCT/EP2024/053192 filed Feb. 8, 2024, which published as PCT Publication No. WO 2024/165676 A1 on Aug. 15, 2024, which claims benefit of European patent application Serial No. PCT/EP2023/053254 filed Feb. 9, 2023.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy was created Aug. 7, 2025, is named Y7954-00668SL.xml and is 9.84 bytes in size.

### FIELD OF THE INVENTION

The present invention relates to the field of plant biotechnology. Specifically, the present invention relates to a melon plant comprising a modified gene which leads to an improved shelf life of melon fruits, as well as to methods for identifying and selecting such a melon plant, and to plant parts, progeny, seed and fruit of such a melon plant. The invention further relates to methods and nucleic acids for producing such a plant, and to methods and genetic markers for selection of such a plant. The invention also relates to a modified gene, and to the use of said gene to improve the shelf life of melon fruits. The invention is also directed to a marker for identification of said modified gene in a melon plant, and to use of said marker.

### BACKGROUND OF THE INVENTION

Melons are the edible fruits of the Cucumis melo plant, which belongs to the Cucurbitaceae family. This family also comprises, for example, cucumber, pumpkin, gherkin, watermelon, and zucchini. Cucumis melo plants (hereafter also referred to as “melon plants”) were among the earliest plants that were domesticated by man, and among the first crop species introduced in the American continent by Europeans. There are many different types of melon fruits, varying in size, shape, color, texture and sweetness. Popular types are, for example, Cantaloupe, Harper, Charentais, Galia, Honeydew and Piel de Sapo.

A very important property of commercially produced melons is their storability after harvest. Melons are typically grown in warm regions, and after harvest they are transported across the globe for consumption. Physiologically, melon plants can be divided into two categories, based on the way in which their fruits ripen. Some melon types are climacteric, while other melon types are non-climacteric. Climacteric fruit ripening is characterized by a sharp increase in respiration rate and in autocatalytic ethylene production, while these two physiological phenomena do not occur during non-climacteric fruit ripening. Climacteric melons generally have a short shelf life (typically between two and five days after harvest), because the ripening process continues after harvest. They are generally also more aromatic than non-climacteric melons, in which ripening stops at the time of harvest. Non-climacteric melons typically have a post-harvest shelf life of between ten and 21 days.

Especially for climacteric melon types it may be challenging to keep the fruits sufficiently fresh between the time of harvest and the time of sale to the consumer. Climacteric fruit ripening is characterized by a respiration burst and the autocatalytic synthesis of ethylene. This triggers a set of biochemical and physiological processes that alter the rind color of the fruit, and the firmness, the flavor, the aroma and the texture of the fruit flesh. An abscission layer is formed at the fruit peduncle, allowing the fruits to fall from the plant upon maturity. Fruit firmness may decrease very rapidly in climacteric genotypes, and this fruit softening negatively affects the quality and post-harvest lifetime of a melon, because it limits its storability and shelf life. This results in considerable commercial losses for growers and retailers. However, consumers prefer the aromatic, fruity flavors of climacteric melons over the less tasty non-climacteric melons. It is therefore highly desirable to provide climacteric melons that have a delayed fruit ripening, and, as a result of this, an improved shelf life.

In general, melons of the cantalupensis and reticulatus types show climacteric ripening and short shelf-life, whereas inodorus types such as Piel de Sapo are non-climacteric and show long shelf-life (Saladié et al, 2015, BMC Genomics 16, 440). This division is however not absolute, because the climacteric ripening behavior is a genetically determined trait, that can be introduced into a non-climacteric genetic background. For example, a climacteric Piel de Sapo melon has been developed through plant breeding efforts (Ríos et al, 2017, Plant J 91, p. 671-683).

It has become clear that fruit ripening is a genetically very complex trait. In various studies, multiple genes and Quantitative Trait Loci (QTLs) have been identified that play a major or minor role in the climacteric ripening process (see for example Ayub et al, 1996, Nature Biotech 14, p. 862-866; Pereira et al, 2020, Hort Res 7, 187; Ríos et al, 2017, Plant J 91, p. 671-683). Research in tomato, the traditional model species for studies on climacteric fruit ripening, has led to the identification of various transcription factors that control the biosynthetic pathway of ethylene in fleshy fruits (see for example Pech et al, 2013, Curr Agricultural Sci Tech 19, p. 1-18; Cherian et al, 2014, J Exp Bot 65, p. 4705-4722; Pereira et al, 2020, Hort Res 7, 187). The process of climacteric fruit ripening in melon and tomato appears to have some mechanisms in common, but both genetically and physiologically there are some differences. For that reason, more studies are now also being performed directly in melon, with the important advantage that there are both climacteric and non-climacteric genotypes available within the same species. Most likely, climacteric and non-climacteric pathways for fruit ripening co-exist in melon (Pech et al, 2008, Plant Sci 175, p. 114-120).

In the research leading to the present invention, it was found that the presence of a specific genetic determinant on chromosome 10 of the melon genome causes a delay in the ethylene peak that occurs during climacteric ripening, as is shown in Example 2. This delayed ethylene production results in a delay of the softening of fruit flesh, and hence in an improved shelf life of melon fruits that normally (in the absence of said genetic determinant) have a climacteric ripening behavior and a limited shelf life. This research further revealed that said genetic determinant is a gene that is transcribed as a long non-coding RNA (lncRNA), as is illustrated in Example 1. The trait of the invention is transmitted genetically in a monogenic, intermediate fashion. “Intermediate inheritance” is also known as incompletely dominant inheritance, in which the phenotype of the F1 progeny is intermediate between the two (more contrasting, extreme) phenotypes of both parents.

Citation or identification of any document in this application is not an admission that such document is available as prior art to the present invention.

### SUMMARY OF THE INVENTION

According to one aspect of the present invention, the present invention relates to a Cucumis melo plant comprising a modified lncRNA gene, the wild type of which has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene.

In some embodiments, the modification is in the second exon of the modified lncRNA gene.

In some embodiments, the modification comprises a G1571A mutation at position 1571 of SEQ ID NO: 1.

In some embodiments, the Cucumis melo plant comprises the G1571A mutation at position 1571 of SEQ ID NO: 1 and has the coding sequence of SEQ ID NO: 2.

In some embodiments, the Cucumis melo plant comprises the G1571A mutation at the corresponding position of the homologous sequence having at least 95% sequence identity to SEQ ID NO: 1.

In some embodiments, the fruit of the Cucumis melo plant having the improved shelf life of the fruits exhibits a phenotype of prolonged firmness of the fruit flesh or a delay of a maximum production level of ethylene during ripening of the fruit.

In some embodiments, the fruit of the Cucumis melo plant having the improved shelf life of the fruits exhibits the phenotype of prolonged firmness of the fruit flesh

In some embodiments, the fruit of the Cucumis melo plant having the improved shelf life of the fruits exhibits the phenotype of the delay of the maximum production level of ethylene during ripening of the fruit.

In some embodiments, the fruit of the Cucumis melo plant having the improved shelf life of the fruits exhibits the phenotype of prolonged firmness of the fruit flesh and the delay of the maximum production level of ethylene during ripening of the fruit.

In some embodiments, the Cucumis melo plant comprises fruit that is a climacteric melon.

In some embodiments, the modified lncRNA gene is as comprised in the genome of a Cucumis melo plant, representative seed of which was deposited with the NCIMB under deposit number NCIMB 43973.

In some embodiments, the modified lncRNA gene is introgressed from a plant grown from the seed deposited with the NCIMB under deposit number NCIMB 43973 or from a progeny plant thereof.

In some embodiments, the modified lncRNA gene is homozygously present.

In some embodiments, the modified lncRNA gene is heterozygously present.

According to another aspect of the present invention, the present invention relates to a marker for the identifying a Cucumis melo plant having a modified lncRNA gene, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene;
wherein the marker has the sequence of SEQ ID NO: 3 or a part thereof capable of detecting the G1571A mutation;
wherein the marker is capable of distinguishing between the G1571A mutation and the wildtype sequence.

According to another aspect of the present invention, the present invention further relates to a method for identifying a Cucumis melo plant having improved shelf life comprising determining the presence of the marker as described herein in the Cucumis melo plant.

In another aspect of the present invention, the present invention relates to a seed of, or from, or that produces the Cucumis melo plant as described herein that comprises the modified lncRNA gene.

In yet another aspect of the present invention, the present invention further relates to propagation material from or suitable for producing the Cucumis melo plant as described herein, wherein the propagation material is suitable for sexual reproduction, and a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem, and wherein the propagation material comprises the modified lncRNA gene.

According to another aspect of the present invention, the present invention relates to method of producing a Cucumis melo plant comprising a modified lncRNA gene, comprising introducing the modified lncRNA gene in to the genome of the Cucumis melo plant, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene.

In yet another aspect of the present invention, the present invention further relates to a method of selecting a Cucumis melo plant comprising a modified lncRNA gene, comprising:
a) identifying by genetic analysis the presence of the modified lncRNA gene in the genome of the Cucumis melo plant,
b) selecting a plant that comprises the modified lncRNA gene;
c) optionally further testing a fruit of the selected plant for improved shelf life as compared to fruit an isogenic Cucumis melo plant lacking the modified lncRNA gene, and
d) optionally selecting the further tested plant whose fruit exhibits improved shelf life as compared to fruit of an isogenic Cucumis melo plant lacking the modified lncRNA gene;
wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

In some embodiments, the method comprises the steps of:
c) further testing a fruit of the selected plant for improved shelf life as compared to fruit an isogenic Cucumis melo plant lacking the modified lncRNA gene, and
d) selecting the further tested plant whose fruit exhibits improved shelf life as compared to fruit of an isogenic Cucumis melo plant lacking the modified lncRNA gene.

In another aspect of the present invention, the present invention relates to a method for producing a Cucumis melo plant, comprising the steps of:
(a) crossing a first Cucumis melo parent plant whose genome comprises a modified lncRNA gene with a second Cucumis melo parent plant to obtain an F1 population;
(b) optionally performing one or more rounds of selfing and/or crossing with a F1 Cucumis melo plant to obtain a further generation population;
(c) selecting from the population resulting from the cross of step a) or from the further generation population of step b), a Cucumis melo plant that comprises the modified lncRNA gene;
wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

In some embodiments, the plant is grown from a seed deposited under NCIMB accession number 43973, or a progeny plant thereof that has retained the modified lncRNA gene.

In yet another aspect of the present invention, the present invention further relates to a method for the production of hybrid Cucumis melo seed comprising:
crossing a first Cucumis melo parent plant with a second Cucumis melo parent plant, and harvesting the hybrid seed;
wherein the first Cucumis melo parent plant or the second Cucumis melo parent plant comprises a modified lncRNA gene, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

The invention further relates to a hybrid Cucumis melo seed produced by the method as described herein.

In some embodiments of the method for producing the hybrid seed, the second Cucumis melo parent plant comprises the modified lncRNA gene.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### Deposits

Seeds of Cucumis melo population T_21R.X_21385, comprising the improved shelf-life trait of the invention homozygously on chromosome 10, were deposited with NCIMB Ltd, Wellheads Place, Dyce, Aberdeen AB21 7 GB United Kingdom on 13 Apr. 2022 under deposit accession number NCIMB 43973 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, can best be understood in conjunction with the accompanying drawings.

FIG. 1 shows the significant delay in endogenous ethylene production (as measured in vpm, volumes of ethylene per million volumes of air) in a melon fruit produced by plants harboring the trait of the invention (solid line), as compared to a melon fruit produced by isogenic plants not harboring the trait of the invention (dashed line).

FIG. 2 shows how the trait of the invention affects endogenous ethylene production in an F1 hybrid background, resulting from crossing a mother line harboring the trait of the invention (solid line) to a father line not carrying the trait of the invention (dashed line). The F1 hybrid plants produce melon fruits in which the endogenous ethylene peak is intermediate in its timing (dotted line), when compared to the peaks in both parental lines.

FIG. 3 shows fruit flesh firmness of melon fruits (as measured in kg with a handheld Facchini FT327 penetrometer with an 11 mm cone) of plants harboring the trait of the invention (left bar), as compared to the melon fruits of isogenic plants not harboring the trait of the invention (right bar). The trait of the invention causes melon fruits to remain much firmer after harvest.

FIG. 4 shows how the trait of the invention affects the firmness of melon fruits in an F1 hybrid background, resulting from crossing a mother line harboring the trait of the invention (left bar) to a climacteric father line not carrying the trait of the invention (middle bar). On average melon fruits produced by the F1 hybrid plants (right bar) are only slightly harder than those of the father line, and much softer than the fruits produced by the mother line.

### DETAILED DESCRIPTION OF THE INVENTION

This invention relates to a Cucumis melo plant producing fruits with an improved shelf life. A melon fruit that is stored after harvest (rather than being consumed immediately after harvest) will gradually become less appealing to the consumer. The factors that contribute most to the decreased consumer appeal are fruit flesh softening and a deterioration of the taste.

The term “shelf life” relates to the period of time during which a melon fruit can be stored before it is considered unsuitable for sale or consumption. Shelf life is preferably assessed during storage, based on various characteristics of the fruit, such as the rind color, flesh color, firmness, aroma and/or sugar content of the fruit flesh. Not only does improved shelf life provide more flexibility in transporting harvested melons, it also provides more flexibility in the harvesting process itself. Traditionally, because individual melon fruits on any given plant ripen at different moments in time, a melon grower in a commercial setting needs to harvest fruits from his crop every single day, in order to avoid individual fruits to become overripe and commercially unattractive. Melon plants that produce fruits with an improved shelf life provide the grower with more flexibility: their fruits can be left on the plant for several days past the traditional harvesting time, such that the grower is able to harvest many fruits at the same time, without jeopardizing the overall quality of his melon harvest. The term “improved shelf life” as used in this application thus refers to a prolonged storability both on the plant and after harvest, i.e. to an increased flexibility in harvesting window of melon fruits, and to a prolonged storability after harvest. The latter term is to be interpreted as a significant increase in the storability of a climacteric melon fruit after it has been harvested from a plant of the invention (measured in days after harvest), as compared to climacteric melon fruits from a control plant. Plants of the invention, having improved shelf life, display a delay of the ethylene peak during ripening of melon fruits produced by said plant, and they also display a delay of fruit flesh softening in melon fruits produced by said plant. The term “improved shelf life” can thus also be defined as displaying a delay of the ethylene peak during ripening of the fruits of the melon plant.

The timing of the endogenous ethylene peak is typically measured in days after pollination, by monitoring the ethylene concentration at regular intervals in at least ten fruits. Detecting the endogenous ethylene peak during melon fruit ripening can be done by the method described by Pereira et al (2017, The Plant J 91, p. 172-183).

In one embodiment, the average timing of the onset of the endogenous ethylene peak in fruits produced by a plant of the invention having improved shelf life is delayed by at least three days, by at least four days, by at least five days, by at least six days, by at least seven days, by at least eight days, by at least nine days, by at least ten days, by at least eleven days, by at least twelve days, by at least 13 days, by at least 14 days, by at least 15 days, by at least 16 days, by at least 17 days, by at least 18 days, by at least 19 days, or by at least twenty days, when compared to the average timing of the onset of the endogenous ethylene peak in fruits produced by a control plant. The onset of the endogenous ethylene peak is the moment at which the ethylene can first be detected.

Another reference point that can be used is the actual peak in endogenous ethylene production, i.e. the number of days after pollination at which the endogenous ethylene production reaches its maximum value as compared to the control plant. In one embodiment, the average timing of the endogenous ethylene peak in fruits produced by a plant of the invention having improved shelf life is delayed by at least three days, by at least four days, by at least five days, by at least six days, by at least seven days, by at least eight days, by at least nine days, by at least ten days, by at least eleven days, by at least twelve days, by at least 13 days, by at least 14 days, by at least 15 days, by at least 16 days, by at least 17 days, by at least 18 days, by at least 19 days, or by at least twenty days, when compared to the average timing of the endogenous ethylene peak in fruits produced by a control plant.

A “control plant” is intended to mean a melon plant that has the same genetic background as the cultivated melon plant of the present invention, wherein the control plant does not have the modified gene of the invention that leads to improved shelf life of the fruits. Not having the modified gene is intended to mean that the modification is not present in the gene. The control plant thus has the non-modified gene. When used in comparison with the plant of the invention, the control plant is grown for the same length of time and under the same conditions as the cultivated melon plant of the invention. Thus, a control plant may be a near-isogenic line, an inbred line or a hybrid, provided that they have the same genetic background as the melon plant of the present invention, except that it does not have the modified gene of the invention that leads to improved shelf life of the fruits.

This invention relates to a Cucumis melo lncRNA gene according to SEQ ID NO: 1, or according to a sequence having at least 50% sequence identity to SEQ ID NO: 1, having a modification, wherein the modification comprises an insertion, a deletion or a substitution. It should be understood that the insertion, deletion or substitution that is present in said gene is not included in said at least 50% sequence identity, because it is an essential feature of the claimed invention. This invention also relates to a Cucumis melo sequence having at least 60% sequence identity, at least 70% sequence identity, at least 80% sequence identity, at least 90% sequence identity, at least 95% sequence identity, at least 96% sequence identity, at least 97% sequence identity, at least 98% sequence identity, or at least 99% sequence identity to SEQ ID NO: 1, having a modification, wherein the modification comprises an insertion, a deletion or a substitution. It should be understood that the insertion, deletion or substitution that is present in said gene is not included in said percentages of sequence identity, because it is an essential feature of the claimed invention.

The term “at least 50% sequence identity” as used henceforth in this application is always intended to also encompass at least 60% sequence identity, at least 70% sequence identity, at least 80% sequence identity, at least 90% sequence identity, at least 95% sequence identity, at least 96% sequence identity, at least 97% sequence identity, at least 98% sequence identity, or at least 99% sequence identity.

The Cucumis melo gene of the invention is transcribed as a long non-coding RNA (lncRNA) transcript comprising two exons. Long non-coding RNA molecules are a type of RNA, generally defined as transcripts of at least 200 nucleotides that are not translated into protein. They are functional molecules that are usually involved in transcriptional control or in post-transcriptional regulation of gene expression (for example in splicing and/or translation of RNA molecules), and they have been shown to be regulators of various agronomically important traits in plants, such as phosphate starvation response, flowering time and interaction with symbiotic organisms. For most annotated lncRNAs no biological function or mode of action has so far been identified.

When compared to messenger RNA (mRNA) molecules that are translated into protein sequences, lncRNAs are generally expressed at a lower abundance, and they exhibit a higher tissue specificity and developmental stage specificity.

The term “modified” relates to a modification of said Cucumis melo lncRNA gene, which modification (an insertion, a deletion or a substitution in the sequence according to SEQ ID NO: 1, or a sequence having at least 50% sequence identity to SEQ ID NO: 1) causes the lncRNA transcript encoded by the modified lncRNA gene to have a changed function, or a reduced function, or which causes it to be non-functional.

Preferably, the modified lncRNA gene of the invention comprises a modification in the second exon. In one embodiment, the modified lncRNA gene of the invention comprises a G1571A mutation at position 1571 in SEQ ID NO: 1. The term “G1571A mutation” refers to the substitution of a guanine at position 1571 in said sequence by an adenine. In a further embodiment, said modified gene comprises the sequence of SEQ ID NO: 2.

This invention also relates to a Cucumis melo plant comprising a lncRNA gene according to SEQ ID NO: 1, or according to a sequence having at least 50% sequence identity to SEQ ID NO: 1, having a modification, wherein the modification comprises an insertion, a deletion or a substitution, which modified gene leads to improved shelf life of the fruits, when compared to an isogenic melon plant lacking the modification.

The improved shelf life of the fruits is characterized by a prolonged firmness of the fruit flesh, and/or by a delay of the ethylene peak during ripening of the fruits.

Said modification causes the lncRNA transcript encoded by the modified lncRNA gene to have a changed function, or a reduced function, or causes it to be non-functional. In one embodiment, the modified gene comprises a modification in the second exon. In a further embodiment, the modified lncRNA gene of the invention comprises a G1571A mutation at position 1571 in SEQ ID NO: 1. In a further embodiment, said modified gene comprises the sequence of SEQ ID NO: 2.

Preferably, the Cucumis melo plant of the invention shows climacteric fruit ripening. In one embodiment, the Cucumis melo plant of the invention comprises a lncRNA gene with a modification, wherein the modified gene is as present in the genome of a Cucumis melo plant, representative seed of which was deposited with the NCIMB under deposit number NCIMB 43973. The invention also relates to a Cucumis melo plant comprising a lncRNA gene with a modification, wherein the modified gene is introgressed from NCIMB 43973 or from a progeny plant thereof. Preferably, the melon plant into whose genome the lncRNA gene with a modification is introgressed shows climacteric fruit ripening. Suitably, it is selected from Cantaloupe, Harper, Honeydew, Galia, Ananas, Charentais, Edible Skin, or any other melon type that shows climacteric fruit ripening, including climacteric Piel de Sapo.

The invention also relates to a marker for the identification of a modified Cucumis melo lncRNA gene according to the invention, wherein the marker is capable of distinguishing between the G1571A mutation and the wildtype sequence, in particular a marker comprising SEQ ID NO: 3 or a part thereof which comprises the G1571A mutation. It should be noted that SEQ ID NO: 3 corresponds to the positive strand of the genomic sequence that comprises the lncRNA gene of the invention, and because it is complementary to a part of the sequence of SEQ ID NO: 1 and SEQ ID NO: 2, it comprises a C>T mutation, which corresponds to the G1571A mutation on the negative strand.

A marker can be defined as a reference sequence that comprises the modification(s) that can be detected using any suitable method known. The term “marker”, “genetic marker” or “DNA marker” refers to a feature of an individual's genome (e.g. a nucleotide or a polynucleotide sequence that is present in an individual's genome) that is associated with one or more loci of interest. In some embodiments, a genetic marker is polymorphic in a population of interest. Genetic markers include, for example, single nucleotide polymorphisms (SNPs), indels (i.e. insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic markers can, for example, be used to locate genetic loci containing alleles on a chromosome that contribute to variability of phenotypic traits. The term “marker” or “genetic marker” can also refer to a polynucleotide sequence complementary to a genomic sequence, such as a sequence of a nucleic acid used as a probe. The term “marker” then refers to a physical entity that can be used in molecular biological techniques for detecting the mutation.

In the context of the present invention, a marker allows unambiguous detection of the genetic basis of the improved shelf-life trait, and the selection of plants that harbor the improved shelf-life trait at any stage of their life cycle, even when the plants are not yet bearing fruits, e.g. during the seedling stage. Marker-assisted breeding and selection greatly increases the speed with which a trait can be introduced into different genetic backgrounds, and with which it can be commercialized.

Methods for detecting markers and specific alleles are abundantly known in the field. In general, these methods allow to distinguish between two different alleles of a marker, on a specific chromosome. Detection of a polymorphism can be achieved by electrophoretic techniques, but the widespread availability of DNA sequencing often makes it easier to simply sequence amplified products directly. Once the polymorphic sequence difference is known, rapid assays for the detection of a polymorphism can be designed for progeny testing, generally involving some version of PCR amplification of specific alleles.

In particular examples, PCR detection and quantification is carried out using two labeled fluorogenic oligonucleotide forward primers and an unlabeled common reverse primer, for example, KASP™ (KBiosciences). Suitable primers for the detection of the G1571A mutation are, for example, the primers of SEQ ID NO: 4 (forward primer for the wildtype allele), SEQ ID NO: 5 (forward primer for the mutant allele), and SEQ ID NO: 6 (common reverse primer).

This invention further relates to the use of a marker for the identification of said modified gene, in particular to the use of a marker comprising SEQ ID NO: 3 or a part thereof which comprises the G1571A mutation, or SEQ ID NO: 3.

This invention also relates to the use of a marker for the identification of a Cucumis melo plant producing fruits with an improved shelf life, in particular a marker comprising SEQ ID NO: 3 or a part thereof which comprises the G1571A mutation. The invention further relates to the use of a marker in the development of a Cucumis melo plant producing fruits with an improved shelf life, in particular a marker comprising SEQ ID NO: 3 or a part thereof which comprises the G1571A mutation.

This invention also relates to a melon seed comprising a lncRNA gene according to SEQ ID NO: 1 having a modification, or a sequence having at least 50% sequence identity to SEQ ID NO: 1 having a modification, wherein the melon plant grown from said seed produces fruits with an improved shelf life when compared to an isogenic melon plant lacking the modification, as a result of the presence of the modified gene. In one embodiment, said melon seed comprises a lncRNA gene comprising SEQ ID NO: 2.

The current invention also relates to propagation material capable of developing into and/or being derived from a Cucumis melo plant comprising a lncRNA gene according to SEQ ID NO: 1, or according to a sequence having at least 50% sequence identity to SEQ ID NO: 1, having a modification, wherein the modification comprises an insertion, a deletion or a substitution of at least one nucleotide, which modified gene leads to improved shelf life of the fruits, when compared to an isogenic melon plant lacking the modification.

Preferably, said propagation material is suitable for sexual reproduction, and is in particular selected from a microspore, pollen, an ovary, an ovule, an embryo sac and an egg cell, or is suitable for vegetative reproduction, and is in particular selected from a cutting, a root, a stem cell, and a protoplast, or is suitable for tissue culture of regenerable cells or protoplasts, which regenerable cells or protoplasts are in particular selected from a leaf, pollen, an embryo, a cotyledon, a hypocotyl, a meristematic cell, a root, a root tip, an anther, a flower and a stem, and wherein the propagation material comprises a lncRNA gene according to SEQ ID NO: 1, or according to a sequence having at least 50% sequence identity to SEQ ID NO: 1, having a modification, wherein the modification comprises an insertion, a deletion or a substitution of at least one nucleotide, which modified gene leads to improved shelf life of the fruits, when compared to an isogenic melon plant lacking the modification. In one embodiment, said propagation material comprises a lncRNA gene comprising SEQ ID NO: 2.

In one embodiment, said propagation material is derived from a Cucumis melo plant, representative seed of which was deposited with the NCIMB under deposit number NCIMB 43973, or from a progeny plant thereof.

The invention also relates to progeny of a plant, a cell, a tissue, or a seed of the invention, which progeny comprises the modified lncRNA gene of the invention as defined herein, the presence of which modified lncRNA gene leads to improved shelf life of the fruits. Such progeny can in itself be a plant, a cutting, a seed, a cell, or a tissue.

As used herein, ‘progeny’ is intended to mean the first and all further descendants, such as an F1, F2, or further generation, from a cross with a plant of the invention, wherein a cross comprises a cross with itself or a cross with another plant, and wherein a descendant that is determined to be progeny comprises the modified lncRNA gene of the invention as defined herein that leads to improved shelf life of the fruits. The plant of the invention that is used in this cross is optionally a plant grown from seed of deposit NCIMB 43973, or from progeny seed thereof which is a direct or further descendant through crossing a plant grown from the deposited seed with itself or with another plant for one or more subsequent generations, wherein the progeny seed has retained the modified lncRNA gene of the invention.

Progeny also encompasses a C. melo plant that carries the modified lncRNA gene of the invention and has the improved shelf life trait, and is obtained from the plant, or progeny of a plant, of the invention by vegetative propagation or another form of multiplication.

The present invention further relates to a method for identifying a melon plant that produces fruits with an improved shelf life, wherein the method comprises screening a melon plant population for the presence of a lncRNA gene according to SEQ ID NO: 1 having a modification, or a sequence having at least 50% sequence identity to SEQ ID NO: 1 having a modification, and identifying the melon plant that comprises at least one modified allele of said modified gene as a melon plant that produces fruits with an improved shelf life. In one embodiment, the invention relates to a method for identifying a melon plant that produces fruits with an improved shelf life, wherein the method comprises screening a melon plant population for the presence of a G1571A mutation at position 1571 in SEQ ID NO: 1. The presence of one modified allele would be sufficient to confer the mutant phenotype onto a plant, considering the fact that the trait of the invention inherits in an intermediate fashion. Optionally, said method may also comprise the step of phenotypically screening for the presence of the improved shelf life trait. Furthermore, said method may also comprise the step of selecting the melon plant that produces fruits with an improved shelf life.

The present invention also relates to a method for producing a melon plant that produces fruits with an improved shelf life, comprising the step of introducing a modification in the lncRNA gene according to SEQ ID NO: 1 or according to a sequence having at least 50% sequence identity to SEQ ID NO: 1 by random mutagenesis or site-directed mutagenesis, wherein the modification comprises an insertion, a deletion or a substitution of at least one nucleotide, and wherein the modification results in an improved shelf life when compared to an isogenic melon plant lacking the modification. In one embodiment, the modification comprises a modification in the second exon, preferably a G1571A mutation at position 1571 in SEQ ID NO: 1. Said improved shelf life is characterized by a delay of the ethylene peak during ripening of melon fruits produced by said plant, and/or in a delay of fruit flesh softening in melon fruits produced by said plant.

This invention also relates to a method for producing a melon plant that produces fruits with an improved shelf life, said method comprising:
a) crossing a melon plant comprising a modified gene comprising an insertion, a deletion or a substitution of at least one nucleotide in the sequence of SEQ ID NO: 1, or in a sequence having at least 50% sequence identity to SEQ ID NO: 1, with another melon plant to obtain an F1 population;
b) optionally performing one or more rounds of selfing and/or crossing a melon plant from the F1 to obtain a further generation population; and
c) selecting from the population a melon plant that comprises the modified gene and that produces fruits with an improved shelf life.

In one embodiment, the crossing of step a) is between two plants of the same melon type, for example two Cantaloupe-type melon plants, or two Harper-type melon plants. In another embodiment, the crossing of step a) is between two plants of a different melon type, for example a Cantaloupe-type melon plant and a Honeydew-type melon plant, or a Harper-type melon plant and a Honeydew-type melon plant. The trait of the invention can be crossed into melon plants regardless of their genetic background, but especially when crossed into a climacteric melon genotype (such as Cantaloupe, Honeydew, Harper, Galia, Ananas, Charentais, Edible Skin, or any other melon type that shows climacteric fruit ripening, including climacteric Piel de Sapo) its phenotypic effect will be significant. This is illustrated in Example 3.

The invention further relates to hybrid Cucumis melo seed and to a method for producing said hybrid Cucumis melo seed, comprising crossing a first Cucumis melo parent plant with a second Cucumis melo parent plant and harvesting the resultant hybrid Cucumis melo seed, wherein the first parent plant and/or the second parent plant comprises the modified lncRNA gene of the invention. The resulting hybrid seed, and the hybrid plant producing fruits with an improved shelf life that can be grown from the hybrid seed, is also a part of the invention. In a preferred embodiment, one of the parent plants comprises the modified lncRNA gene of the invention homozygously.

This invention also relates to a method of growing a Cucumis melo plant comprising a lncRNA gene according to SEQ ID NO: 1, or according to a sequence having at least 50% sequence identity to SEQ ID NO: 1, having a modification, wherein the modification comprises an insertion, a deletion or a substitution, which modified gene leads to improved shelf life of the fruits, when compared to an isogenic melon plant lacking the modification.

This invention further relates to a method of growing a Cucumis melo comprising a lncRNA gene with a modification, wherein the modified gene is as present in the genome of a Cucumis melo plant, representative seed of which was deposited with the NCIMB under deposit number NCIMB 43973. The invention also relates to a method of growing a Cucumis melo plant comprising a lncRNA gene with a modification, wherein the modified gene is introgressed from NCIMB 43973 or from a progeny plant thereof. The invention also relates to a method of growing a Cucumis melo plant comprising a lncRNA gene with a G1571A mutation at position 1571 in SEQ ID NO: 1. Preferably, the melon plant into whose genome the lncRNA gene with a modification is introgressed shows climacteric fruit ripening. Suitably, it is selected from Cantaloupe, Harper, Honeydew, Galia, Ananas, Charentais, Edible Skin, or any other melon type that shows climacteric fruit ripening, including climacteric Piel de Sapo.

The invention further relates to a method for the production of a plant comprising the modified lncRNA gene of the invention, by using tissue culture or by using vegetative propagation.

The invention further provides a method for the production of a plant comprising the modified lncRNA gene of the invention by using a doubled haploid generation technique to generate a doubled haploid line that is completely homozygous, and therefore homozygously comprises the modified lncRNA gene of the invention, and that produces fruits with an improved shelf life.

The invention further relates to a method for the production of a plant comprising the modified lncRNA gene of the invention, wherein the presence of said modified lncRNA gene leads to improved shelf life of the fruits, which method comprises growing a seed comprising said modified lncRNA gene into the said plant.

The invention further relates to a cell comprising the modified lncRNA gene of the invention as defined herein. A cell of the invention can be obtained from, or be present in, a plant of the invention. Such a cell may either be in isolated form, or a part of the complete plant, or from a part thereof, and still constitutes a cell of the invention because such a cell comprises the genetic information that determines the modified lncRNA gene as described herein. Each cell of a plant of the invention carries the modified lncRNA gene of the invention, and thereby the genetic information that leads to improved shelf life of the fruits. A cell of the invention may also be a regenerable cell that can regenerate into a new plant of the invention. The presence of the genetic information in this context is the presence of the modified lncRNA gene of the invention, wherein the modified lncRNA gene is as defined herein.

### EXAMPLES

### Example 1

### Mapping of the Improved Shelf-Life Trait on Chromosome 10

A phenotypic screen was performed on a population of melon plants of the Cantaloupe type. The aim of this screen was to identify melon plants of which the rind of the melon fruits remained green for a longer period of time, while the fruit rind of melons produced by other Cantaloupe plants changed from green to yellow. The screen was successful, and melon plant Me5.140 was selected. This plant showed a clearly delayed yellowing of the fruit rind, which is a phenotypic marker that is correlated to fruit ripening.

A mapping project was then initiated to elucidate which genetic variation caused the delayed fruit rind yellowing in plant Me5.140. This plant was first crossed to a climacteric (aromatic) reference line named GBN831, and an F1 plant from this cross was subsequently backcrossed three times to Me5.140, and subsequently inbred for five generations, to obtain a F5BC3 population of about 200 introgression lines. These lines were nearly isogenic to Me5.140, but they harbored small introgression fragments from GBN831 in their genome. This population was grown to maturity in a greenhouse, and at four time points the produced fruits were examined for early yellowing. Five lines were selected which showed a typical climacteric fruit ripening, as scored by their early yellowing of the fruits in comparison to all other lines of the population, which indicated that in these five lines the delayed fruit ripening trait in the Me5.140 genome had been replaced by the corresponding wildtype sequence from the GBN831 line.

One of the five lines was selected, and this line was backcrossed to Me5.140 for further fine-mapping of the trait in the F2 and F3 generations. The plants were screened for the delayed fruit rind yellowing trait, and marker analysis was performed in order to identify markers that were genetically linked to the improved shelf-life trait. An additional phenotypic assay—measurement of fruit firmness at the time of harvest (as measured in kg with a handheld Facchini FT327 penetrometer with an 11 mm cone)—was performed to unambiguously determine whether individual melon plants produced fruits with a limited shelf-life or fruits with an improved (i.e. long) shelf-life. In this population this trait segregated, with aromatic melon fruits (with early color change from green to yellow) having an average fruit firmness of 3.7 at the time of harvest, whereas improved shelf-life melon fruits (with a delayed color change from green to yellow) had an average firmness of 6.4 at the time of harvest.

After several rounds of fine-mapping, a specific SNP (Single Nucleotide Polymorphism) mutation that was perfectly correlated with the improved shelf-life phenotype could be identified as the causal mutation for this trait. When looking into the annotation of this region of chromosome 10, it was observed that the causal SNP mutation was located in the second exon of a long non-coding RNA (lncRNA) gene on chromosome 10. With reference to GenBank sequence LOC107990782, it was a G>A mutation located on position 1571. Therefore it was subsequently named G1571A. The complete wildtype sequence of this gene is comprised in SEQ ID NO: 1, and the complete mutant sequence is comprised in SEQ ID NO: 2.

The observation that the G1571A mutation is located in the second exon of a lncRNA gene implies that a mutated lncRNA transcript is being expressed in mutant melon plants that display the improved shelf-life trait of the invention. At least two splice variants have been detected for this lncRNA gene, but in both known splice variants the complete second exon is present. This means that the mutated region of the lncRNA is present in all known alternative transcripts of this gene, and that the presence of the G1571A mutation invariably results in the expression of a mutated lncRNA transcript.

### Example 2

### Physiological Characterization of the Improved Shelf-Life Trait

An isogenic melon line was produced by crossing out the G1571A mutation from the genome of a plant of the invention. This isogenic line was then used to further characterize the effect of the G1571A mutation on melon fruit ripening.

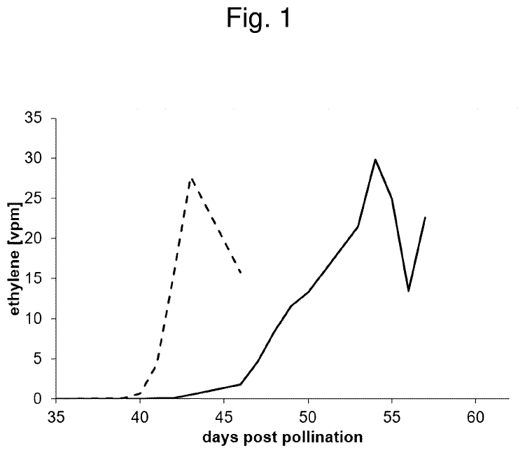

FIG. 1 shows the endogenous production of ethylene in a representative melon fruit (as measured in vpm, volumes of ethylene per million volumes of air) of a plant of the invention (line Me5.140) harboring in its genome a modification in the lncRNA gene (solid line), as compared to the endogenous ethylene production in a melon fruit of an isogenic line (dashed line) from whose genome the mutated lncRNA had been crossed out and replaced by a wildtype sequence. Repeated ethylene measurements were performed on a fruit during ripening at regular intervals until harvest. Gas samples were analyzed using gas chromatography.

In the presence of the modified lncRNA gene the endogenous ethylene production peaked about 54 days after pollination, whereas in the absence of said modification this happened already about 43 days after pollination. In this experiment, we thus observed a delay in the peak of endogenous ethylene production of about eleven days, whereas the onset of the ethylene peak was delayed by about five days. The peak of endogenous ethylene production is a hallmark of climacteric ripening, and this experiment thus demonstrated that the presence of a modification in a specific lncRNA gene results in a significant delay of the onset of climacteric ripening in melon fruits.

In order to further confirm the observed effects of the improved shelf-life trait on a larger number of melon fruits, we then examined a total of 22 fruits of line Me5.140 harboring the trait of the invention (in eleven of which the ethylene production was also measured), and a total of 19 fruits of an isogenic control line lacking the trait of the invention (in ten of which the ethylene production was also measured). No significant differences could be observed in the weight and sweetness of the fruits produced by both lines: fruits from line Me5.140 had an average weight of 1824±311 grams and an average brix value of 11.1±1.7, whereas fruits from the control line had an average weight of 1688±432 grams and an average brix value of 10.1±1.2. However, the effect of the improved shelf-life trait of the invention on fruit flesh firmness was very pronounced: melons produced by line Me5.140 had an average firmness of 5.9±2.3 at the time of harvest (59.3±8.0 days after pollination, on average), whereas melons produced by the isogenic control line were much softer, with an average firmness of 2.3±1.4 at the time of harvest (49.7±3.5 days after pollination, on average). The fruit rind color change from green to yellow occurred much later in line Me5.140: on average at 55.4±6.4 days after pollination, as compared to 42.1±2.8 days after pollination in the isogenic control line.

Measurement of endogenous ethylene production showed that the onset of the ethylene peak came at 45.6±1.0 days after pollination in line Me5.140, and at 40.9±2.3 days after pollination in the isogenic control line. The actual ethylene peak was detected at 52.4±3.0 days after pollination in line Me5.140, and at 43.9±2.2 days after pollination in the isogenic control line.

In this experiment, the presence of the improved shelf-life trait of the invention thus caused a delay in the onset of the ethylene peak of about 4.5 days, a delay in the timing of the actual ethylene peak of about 8.5 days, a delay of about 13 days in the color change of the fruit rind, and a significant delay in fruit flesh softening, without affecting the weight and sweetness of the melon fruits at the time of harvest.

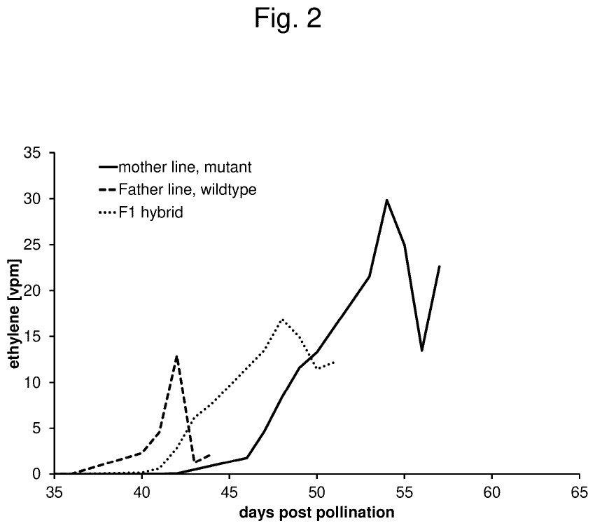

In a subsequent experiment, a melon plant of the invention was pollinated with pollen from a climacteric father line. The mother, father and F1 hybrid lines were then grown alongside each other, and ethylene production was measured in the melon fruits that they produced. FIG. 2 shows that the endogenous ethylene production in melons produced by the father line peaked at 42 days after pollination, and that the ethylene peak in fruits produced by the mother line (harboring the trait of the invention) came 54 days after pollination. Their F1 hybrid showed an ethylene peak that was intermediate in its timing: its endogenous ethylene production peaked 48 days after pollination. From the experiment it became clear that the improved shelf-life trait of the invention is particularly useful when used in the production of hybrids. If one of the parents (in this example the mother line) harbors the improved shelf-life trait of the invention, and this trait is incorporated into a hybrid genome with a single copy, its phenotypic effect of fruit ripening is intermediate. This is consistent with the intermediate inheritance of this trait. Fruit ripening (here measured in terms of the timing of the endogenous ethylene production in a melon fruit) is significantly delayed (in this example by about six days), resulting in melon fruits that ripen more slowly when compared to melon fruits of the climacteric father line.

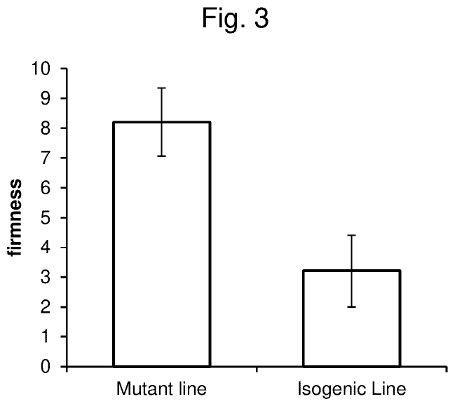

An important factor that determines the commercial usefulness of this trait is its effect on fruit flesh firmness. FIG. 3 shows the fruit flesh firmness of melon fruits (as measured in kg, with a handheld Facchini FT327 penetrometer with an 11 mm cone at the time of harvest) of a plant of the invention harboring in its genome a modification in the lncRNA gene (left bar), as compared to the melon fruits of an isogenic line (right bar) from whose genome the mutated lncRNA had been crossed out and replaced by a wildtype sequence. The graph shows that the fruit flesh of mutant melon fruits of the invention had an average firmness of 8.2, whereas the fruit flesh of fruits from the isogenic line lacking the modification had an average firmness of only 3.2.

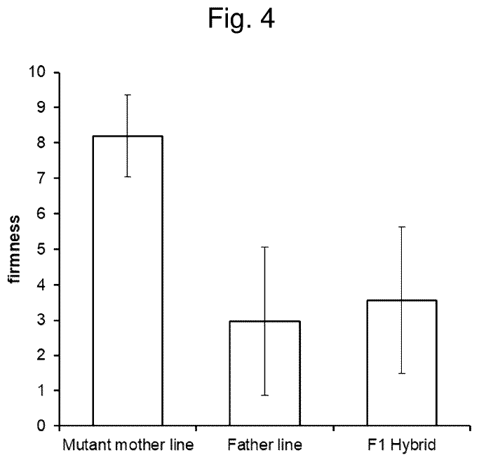

This experiment thus indicated that in the presence of a modification in a specific lncRNA, melon fruits remain much firmer after harvest. To explore the effect of the improved shelf-life trait on fruit firmness in a hybrid context (i.e. when present in a heterozygous state, alongside a wildtype allele), we performed fruit flesh firmness measurements on the mother line, father line and F1 hybrid line that were described above. We observed (FIG. 4) that an F1 hybrid plant produces melon fruits that are only slightly more firm (on average 3.56) at the time of harvest than those of the climacteric father line (on average 2.97), and much less firm than those of the mother line in which the trait of the invention is present in a homozygous state (on average 8.2). This is commercially very interesting, because very firm (i.e. hard) melon fruits are generally not appreciated by consumers, who prefer to eat softer fruits.

In conclusion, when present in a heterozygous state (i.e. in a hybrid genome), the improved shelf-life trait of the invention causes melon fruits to ripen more slowly (as indicated in this experiment by the delay of about six days in the ethylene peak), and that remain soft enough to be appealing to consumers. The invention is therefore commercially very interesting, because it strongly enhances the storability of melon fruits without compromising on its firmness and flavor.

### Example 3

### Introduction of the Improved Shelf-Life Trait in Climacteric Melon Plants

To further explore the commercial potential of the improved shelf-life trait of the invention, an experiment was performed to introduce this trait into climacteric melon plants from an entirely different genetic background. The trait had originally been identified in a Cantaloupe background, and it would be very interesting if it could be used to prolong the shelf-life of other climacteric melon types than Cantaloupe, such as Honeydew, Harper, Galia, Ananas, Charentais, Edible Skin, or any other melon type that shows climacteric fruit ripening, including climacteric Piel de Sapo.

A causal SNP mutation resulting in the trait according to the invention can be identified using the KASP™ (KBiosciences) assay, with two labeled fluorogenic oligonucleotide forward primers and an unlabeled common reverse primer (Table 1).

TABLE 1

| Forward primer | | Common | Wild- | |
|---|---|---|---|---|
| (wildtype | Forward primer | reverse | type | Mutant |
| allele) | (mutant allele) | primer | allele | allele |
| GTATMACCTTTCT | GTATMACCTTTCT | GTCGTTTAATAC | C | T |
| TGTGTTGATAAAA | TGTGTTGATAAAA | AGTTGCTCCAAC | | |
| CATC (SEQ ID | CATT (SEQ ID | TCATT (SEQ | | |
| NO: 4) | NO: 5) | ID NO: 6) | | |

Using this SNP marker suitable for identifying the G1571A mutation, the trait of the invention was crossed into a Honeydew melon background. Two independent Honeydew hybrids harboring a single copy of the introgressed G1571A mutation (i.e. being heterozygous for the trait) were subsequently grown alongside two commercial hybrids of the Honeydew type, namely the climacteric varieties Silver Rock and Silverball. Melon fruits were harvested from each hybrid and stored at 4° C. for a period of 21 days. Fruit firmness was measured at one day after harvest and at 21 days after harvest. Table 2 shows that the two Honeydew hybrids harboring the improved shelf-life (herein abbreviated to “LSL”, for “long shelf-life”) trait of the invention produced melon fruits that retained their firmness throughout the entire storage period (on average 5, both after one day of storage and after 21 days of storage), whereas the firmness of both commercial Honeydew varieties decreased significantly during the three-week storage (on average from 7 to 4 in Silver Rock, and on average from 5 to 3.5 in Silverball). This progressive softening of the fruit flesh is indeed what would be expected for climacteric melons. This experiment therefore indicated that the trait of the invention can also be used in other climacteric melon backgrounds, such as Honeydew, to significantly extend the shelf-life of melon fruits and to prolong their storability without loss of commercial attractiveness. The presence of the trait of the invention ensures that climacteric melon fruits retain their fruit flesh firmness for a much longer period of time.

TABLE 2

| VARIETY | LSL? | 1 DAY | 21 DAYS |
|---|---|---|---|
| Hybrid 1 | yes | 5 | 5, 5 |
| Hybrid 2 | yes | 5 | 5 |
| Silver Rock | no | 7 | 4 |
| Silverball | no | 5 | 3, 5 |

In another experiment, the effect of the improved shelf-life trait of the invention was examined on fruit ripening in melon plants of the Galia type. A Galia-type melon harboring the improved shelf-life trait of the invention (named Me5.019, homozygous for the trait) was compared to a Galia line (named Me1.050) harboring the corresponding wildtype lncRNA sequence. A total of 20 fruits was examined from line Me5.019 (in ten of which the ethylene production was also measured), compared to a total of 23 fruits from line Me1.050 (in eleven of which the ethylene production was also measured). Fruits produced by line Me5.019 weighed on average 1262±232 grams and had an average brix value of 10.3±2.0, whereas fruits produced by line Me1.050 weighed on average 1504±162 grams and had an average brix value of 8.2±1.3, which means that both lines produced melon fruits with a comparable weight and a comparable sweetness. There was, however, a very pronounced difference in fruit flesh firmness at the time of harvest: fruits produced by the control line Me 1.050 had an average firmness of 1.7±1.4 (when harvested 44.5±4.3 days after pollination, on average), whereas fruits produced by line Me5.019 (harboring the improved shelf-life trait of the invention) had an average firmness of 5.3±1.1 (when harvested 60.7±4.1 days after pollination, on average). Measurement of endogenous ethylene production revealed that the onset of the ethylene peak came at 33.8±1.3 days after pollination in control line Me1.050, but only at 53.9±2.9 days after pollination in line Me5.019 (harboring the improved shelf-life trait of the invention). The actual ethylene peak was detected at 36.6±3.9 days after pollination in control line Me1.050, and at 57.7±3.8 days after pollination in line Me5.019 (harboring the improved shelf-life trait of the invention).

The observed effect of the improved shelf-life trait of the invention in a Galia background was thus very pronounced. On average it delayed the ethylene peak (and its onset) by about twenty days. As a result of this, the fruit flesh of fruits produced by Galia plants harboring the trait of the invention remained much firmer than the fruit flesh of fruits produced by Galia plants lacking the trait of the invention, without affecting the weight and sweetness of the melon fruits at the time of harvest.

The invention is further described by the following numbered paragraphs:

1. A Cucumis melo plant comprising a modified lncRNA gene, the wild type of which has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene.

2. The Cucumis melo plant of paragraph 1, wherein the modification is in the second exon of the modified lncRNA gene.

3. The Cucumis melo plant of paragraph 1, wherein the modification comprises a G1571A mutation at position 1571 of SEQ ID NO: 1.

4. The Cucumis melo plant of paragraph 1, comprising the G1571A mutation at position 1571 of SEQ ID NO: 1 and has the coding sequence of SEQ ID NO: 2.

5. The Cucumis melo plant of paragraph 1, comprising the G1571A mutation at the corresponding position of the homologous sequence having at least 95% sequence identity to SEQ ID NO: 1.

6. The Cucumis melo plant of paragraph 1, wherein the fruit of the Cucumis melo plant having the improved shelf life of the fruits exhibits a phenotype of prolonged firmness of the fruit flesh or a delay of a maximum production level of ethylene during ripening of the fruit.

7. The Cucumis melo plant of paragraph 1, comprising fruit that is a climacteric melon.

8. The Cucumis melo plant of paragraph 1, wherein the modified lncRNA gene is as comprised in the genome of a Cucumis melo plant, representative seed of which was deposited with the NCIMB under deposit number NCIMB 43973.

9. The Cucumis melo plant of paragraph 8, wherein the modified lncRNA gene is introgressed from a plant grown from the seed deposited with the NCIMB under deposit number NCIMB 43973 or from a progeny plant thereof.

10. The Cucumis melo plant of paragraph 1, wherein the modified lncRNA gene is homozygously present.

11. The Cucumis melo plant of paragraph 1, wherein the modified lncRNA gene is heterozygously present.

12. A marker for the identifying a Cucumis melo plant having a modified lncRNA gene, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene;
wherein the marker has the sequence of SEQ ID NO: 3 or a part thereof capable of detecting the G1571A mutation;
wherein the marker is capable of distinguishing between the G1571A mutation and the wildtype sequence.

13. A method for identifying a Cucumis melo plant having improved shelf life comprising determining the presence of the marker of paragraph 12 in the Cucumis melo plant.

14. A seed of, or from, or that produces the Cucumis melo plant of paragraph 1 and comprises the modified lncRNA gene.

15. Propagation material from or suitable for producing the Cucumis melo plant of paragraph 1, wherein the propagation material is suitable for sexual reproduction, and a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem, and wherein the propagation material comprises the modified lncRNA gene.

16. A method of producing a Cucumis melo plant comprising a modified lncRNA gene, comprising introducing the modified lncRNA gene in to the genome of the Cucumis melo plant, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene.

17. A method of selecting a Cucumis melo plant comprising a modified lncRNA gene, comprising:
a) identifying by genetic analysis the presence of the modified lncRNA gene in the genome of the Cucumis melo plant,
b) selecting a plant that comprises the modified lncRNA gene;
c) optionally further testing a fruit of the selected plant for improved shelf life as compared to fruit an isogenic Cucumis melo plant lacking the modified lncRNA gene, and
d) optionally selecting the further tested plant whose fruit exhibits improved shelf life as compared to fruit of an isogenic Cucumis melo plant lacking the modified lncRNA gene;
wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

18. The method of paragraph 17 further comprising the steps of:
c) further testing a fruit of the selected plant for improved shelf life as compared to fruit an isogenic Cucumis melo plant lacking the modified lncRNA gene, and
d) selecting the further tested plant whose fruit exhibits improved shelf life as compared to fruit of an isogenic Cucumis melo plant lacking the modified lncRNA gene.

19. A method for producing a Cucumis melo plant, comprising the steps of:
(a) crossing a first Cucumis melo parent plant whose genome comprises a modified lncRNA gene with a second Cucumis melo parent plant to obtain an F1 population;
(b) optionally performing one or more rounds of selfing and/or crossing with a F1 Cucumis melo plant to obtain a further generation population;
(c) selecting from the population resulting from the cross of step a) or from the further generation population of step b), a Cucumis melo plant that comprises the modified lncRNA gene;
wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

20. The method of paragraph 19, wherein the plant is grown from a seed deposited under NCIMB accession number 43973, or a progeny plant thereof that has retained the modified lncRNA gene.

21. A method for the production of hybrid Cucumis melo seed comprising: crossing a first Cucumis melo parent plant with a second Cucumis melo parent plant, and harvesting the hybrid seed;
wherein the first Cucumis melo parent plant or the second Cucumis melo parent plant comprises a modified lncRNA gene, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

22. The hybrid Cucumis melo seed produced by the method of paragraph 21.

23. The method of paragraph 21, wherein the second Cucumis melo parent plant comprises the modified lncRNA gene.

24. The Cucumis melo plant of paragraph 6, wherein the fruit of the Cucumis melo plant having the improved shelf life of the fruits exhibits the phenotype of prolonged firmness of the fruit flesh

25. The Cucumis melo plant of paragraph 6, wherein the fruit of the Cucumis melo plant having the improved shelf life of the fruits exhibits the phenotype of the delay of the maximum production level of ethylene during ripening of the fruit.

26. The Cucumis melo plant of paragraph 1, wherein the fruit of the Cucumis melo plant having the improved shelf life of the fruits exhibits the phenotype of prolonged firmness of the fruit flesh and the delay of a maximum production level of ethylene during ripening of the fruit.

### SEQUENCE INFORMATION

SEQ ID NO: 1 Genomic fragment of Cucumis melo chromosome 10, comprising the wildtype sequence of the lncRNA gene of the invention that is present in Cucumis melo plants grown from seeds that are deposited under accession number NCIMB 43973. The position of the G1571A SNP mutation is indicated as a G in bold and underlined:

AAATAAAAATATTTACAGTGGATAGAATGGAGGAATTATATAAGAAACGA
GGATACCAATTTGCAACCATTCTTTTATTGATGAAGGAAAGAAAATGAAA
ATACAAATAAAGAGTCCAAATTGGCATATTTGGTGGTAAGGTAAGGTAAG
GTGCATTCACACCAACCATTCTTTTCCAATTTTCCTAATCTTTTACATTT
TTCTCTACTATCCTAAATTAGCTTCAATTTAATTCTTTTTCTTTTCTTTT
TTAATTTGGTTAACCATCTTTTTTAAACTACCTTATGCACTATTTAACTT
TTAAAAAAATTGTTATGAATAGAAAAATATATATATATATTATTTATAAA
ATATAACAAAAACTTAAATAGAAAATAGTTTCCATATTTTGCTACTTTGG
AAAATGTTTCTTAATTTTAATAATATGTGATTTTTTTTATTGATTTATGT
GTGAAGTAAAAAATGAAAAAGAAAATATAGGAATGTATCAGTTTATGTGT
TTCTAAAACTTAGATTAATTTATGCTTCTATTTTTTTTTTTTTTTATTTA
CTAATCTCATGCCAAATTAGTTAAATATTTACAATAACTATATCATGCCG
TGATTTAGTAAATGGTGTAAAGTGAAAAAAAAAATTAAATAAAATTCTAA
TTCTGCTCTCTATGAATTTTTAATACAATTTTTTATAGAAAAATTTAAGT
CCACTAGAAAAGAAGGTATATAGGTAGATGTAGGAAAATCTCTCACTCTA
CGCATCATTTCATAACTCAAAAAAAAAAAAAGAATGAATAAATAAATAGA
ATACTACATCAAATAAACTGTGGTTTTGTTAATATTAACCATAAAAATGT
GCTATATTCATTTTAAGTTACACATTCATTGATAGAATATGTTGATTAAA
AATTCAACGAAAGACAGCTAAATTGCATTAATTTCAGAGAATATGTTATG
GATGTTGTGATCATATTCAAGTTCCAATGGGAAGTTGTCAATGATGAATT
GGATCCGTTAGTGTGTTAAAATTTTATTAATACCACAGATAAACACATTA
TAATTCTGGAAGAAAGCTAGCTTGATTCAATATATAATTAAAGACTTTTT
TTTTCTCTCTCTTTTTTTTTTTTTTTTTTTTTTAATTTTGGAGATTTGAT
TTTTCGTTCACATTATATATGCTCATTCTATGAATATGGTCACAAAACAT
TTAAAGTCAATTTATTTCTTTATTTATCACAAACATAATTTTCCTTTTTG
TTTCCGTACAAAATTAAATCAACTCTCTCCCAATTTTACTAATATCACAC
GTATTTCTTACTACTATAATTTCTCTATCTTTATACACTAACCTTACCAT
ATACTTTTTTTTTTATCTTCAAGATATTTCACCTCCCGGCCTATTATCAC
TTCCTCGATATGACATACAGTCATAATCCGTAAGTGTGCTTCTTTTTATC
TTAGGATCACGTTGGTTTTTGGTTTTTTCTATGACAGTCGTTTAATACAG
TTGCTCCAACTCATTTATCGTGGGACACACTAATATAATCAAATATCTAA
CTTCCACACAGCCAAAACAAGATGTTTTATCAACACAAGAAAGGTGATAC
TTTCAAACGTTCAAACATCATTGTTGTTAATGCCATGTAACACACTACTA
CCTCCGGCTTTCCGTTTACACTTTCTTTTATTTATTTATCTATTTATTTA
AAAGTTTTGAAGAAACGGCTTTTCAATGTATGGTAACATTTTTCATAAAT
TCAAATTAAATTATTGTATTTGAA

SEQ ID NO: 2 Genomic fragment of Cucumis melo chromosome 10, comprising a modified sequence of the lncRNA gene of the invention. The position of the G1571A SNP mutation is indicated as an A in bold and underlined:

AAATAAAAATATTTACAGTGGATAGAATGGAGGAATTATATAAGAAACGA
GGATACCAATTTGCAACCATTCTTTTATTGATGAAGGAAAGAAAATGAAA
ATACAAATAAAGAGTCCAAATTGGCATATTTGGTGGTAAGGTAAGGTAAG
GTGCATTCACACCAACCATTCTTTTCCAATTTTCCTAATCTTTTACATTT
TTCTCTACTATCCTAAATTAGCTTCAATTTAATTCTTTTTCTTTTCTTTT
TTAATTTGGTTAACCATCTTTTTTAAACTACCTTATGCACTATTTAACTT
TTAAAAAAATTGTTATGAATAGAAAAATATATATATATATTATTTATAAA
ATATAACAAAAACTTAAATAGAAAATAGTTTCCATATTTTGCTACTTTGG
AAAATGTTTCTTAATTTTAATAATATGTGATTTTTTTTATTGATTTATGT
GTGAAGTAAAAAATGAAAAAGAAAATATAGGAATGTATCAGTTTATGTGT
TTCTAAAACTTAGATTAATTTATGCTTCTATTTTTTTTTTTTTTTATTTA
CTAATCTCATGCCAAATTAGTTAAATATTTACAATAACTATATCATGCCG
TGATTTAGTAAATGGTGTAAAGTGAAAAAAAAAATTAAATAAAATTCTAA
TTCTGCTCTCTATGAATTTTTAATACAATTTTTTATAGAAAAATTTAAGT
CCACTAGAAAAGAAGGTATATAGGTAGATGTAGGAAAATCTCTCACTCTA
CGCATCATTTCATAACTCAAAAAAAAAAAAAGAATGAATAAATAAATAGA
ATACTACATCAAATAAACTGTGGTTTTGTTAATATTAACCATAAAAATGT
GCTATATTCATTTTAAGTTACACATTCATTGATAGAATATGTTGATTAAA
AATTCAACGAAAGACAGCTAAATTGCATTAATTTCAGAGAATATGTTATG
GATGTTGTGATCATATTCAAGTTCCAATGGGAAGTTGTCAATGATGAATT
GGATCCGTTAGTGTGTTAAAATTTTATTAATACCACAGATAAACACATTA
TAATTCTGGAAGAAAGCTAGCTTGATTCAATATATAATTAAAGACTTTTT
TTTTCTCTCTCTTTTTTTTTTTTTTTTTTTTTTAATTTTGGAGATTTGAT
TTTTCGTTCACATTATATATGCTCATTCTATGAATATGGTCACAAAACAT
TTAAAGTCAATTTATTTCTTTATTTATCACAAACATAATTTTCCTTTTTG
TTTCCGTACAAAATTAAATCAACTCTCTCCCAATTTTACTAATATCACAC
GTATTTCTTACTACTATAATTTCTCTATCTTTATACACTAACCTTACCAT
ATACTTTTTTTTTTATCTTCAAGATATTTCACCTCCCGGCCTATTATCAC
TTCCTCGATATGACATACAGTCATAATCCGTAAGTGTGCTTCTTTTTATC
TTAGGATCACGTTGGTTTTTGGTTTTTTCTATGACAGTCGTTTAATACAG
TTGCTCCAACTCATTTATCGTGGGACACACTAATATAATCAAATATCTAA
CTTCCACACAGCCAAAACAAAATGTTTTATCAACACAAGAAAGGTGATAC
TTTCAAACGTTCAAACATCATTGTTGTTAATGCCATGTAACACACTACTA
CCTCCGGCTTTCCGTTTACACTTTCTTTTATTTATTTATCTATTTATTTA
AAAGTTTTGAAGAAACGGCTTTTCAATGTATGGTAACATTTTTCATAAAT
TCAAATTAAATTATTGTATTTGAA

SEQ ID NO: 3 Genetic SNP marker that is able to identify the presence of the G1571A mutation that underlies the trait of the invention in Cucumis melo plants grown from seeds that are deposited under accession number NCIMB 43973. A genomic fragment of chromosome 10 is presented (positive strand), wherein the position of the aforementioned SNP comprising a change from G to A [G1571A] is indicated as [C/T]. This SNP mutation is present in plants grown from seeds of the said deposit. In this sequence Y refers to any pyrimidine (C or T), and M refers to A or C:

GTGTAAACGGAAAGCCGGAGGYAGTAGTGTGTTACATGGCATTAACAACA
ATGATGTTTGAACGTTTGAAAGTATMACCTTTCTTGTGTTGATAAAACAT
[C/T]TTGTTTTGGCTGTGTGGAAGTTAGATATTTGATTATATTAGTGTG
TCCCACGATAAATGAGTTGGAGCAACTGTATTAAACGACTGTCATAGAAA
AAA

SEQ ID NO: 4 Forward primer that specifically recognizes the wildtype allele of the G1571A polymorphism. Suitably it is labelled with HEX dye and used in a KASP™ marker assay in combination with SEQ ID NO: 5 and SEQ ID NO: 6:

GTATMACCTTTCTTGTGTTGATAAAACATC

SEQ ID NO: 5 Forward primer that specifically recognizes the mutant allele of the G1571A polymorphism. Suitably it is labelled with FAM dye and used in a KASP™ marker assay SEQ ID NO: 4 and SEQ ID NO: 6:

GTATMACCTTTCTTGTGTTGATAAAACATT

SEQ ID NO: 6 Common reverse primer (without a fluorophore label) that can be used in a KASP™ marker assay in combination with SEQ ID NO: 4 and SEQ ID NO: 5, to distinguish between the wildtype and mutant alleles of the G1571A polymorphism:

GTCGTTTAATACAGTTGCTCCAACTCATT

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A Cucumis melo plant comprising a modified lncRNA gene, the wild type of which has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene.

2. The Cucumis melo plant of claim 1, wherein:
a) the modification is in the second exon of the modified lncRNA gene; or
b) the modification comprises a G1571A mutation at position 1571 of SEQ ID NO: 1.

3. (canceled)

4. The Cucumis melo plant of claim 1, comprising the G1571A mutation at position 1571 of SEQ ID NO: 1 and has the coding sequence of SEQ ID NO: 2.

5. (canceled)

6. The Cucumis melo plant of claim 1, wherein the fruit of the Cucumis melo plant having the improved shelf life of the fruits exhibits a phenotype of prolonged firmness of the fruit flesh or a delay of a maximum production level of ethylene during ripening of the fruit; or
the fruit of the Cucumis melo plant having the improved shelf life of the fruits exhibits the phenotype of prolonged firmness of the fruit flesh and the delay of a maximum production level of ethylene during ripening of the fruit; or
the plant comprises fruit that is a climacteric melon.

7. (canceled)

8. The Cucumis melo plant of claim 1, wherein the modified lncRNA gene is as comprised in the genome of a Cucumis melo plant, representative seed of which was deposited with the NCIMB under deposit number NCIMB 43973.

9. The Cucumis melo plant of claim 8, wherein the modified lncRNA gene is introgressed from a plant grown from the seed deposited with the NCIMB under deposit number NCIMB 43973 or from a progeny plant thereof.

10. The Cucumis melo plant of claim 1, wherein the modified lncRNA gene is homozygously present.

11. The Cucumis melo plant of claim 1, wherein the modified lncRNA gene is heterozygously present.

12. A marker for the identifying a Cucumis melo plant having a modified lncRNA gene, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene;
wherein the marker has the sequence of SEQ ID NO: 3 or a part thereof capable of detecting the G1571A mutation;
wherein the marker is capable of distinguishing between the G1571A mutation and the wildtype sequence.

13. A method for identifying a Cucumis melo plant having improved shelf life comprising determining the presence of the marker of claim 12 in the Cucumis melo plant.

14. A seed of, or from, or that produces the Cucumis melo plant of claim 1 and comprises the modified lncRNA gene.

15. Propagation material from or suitable for producing the Cucumis melo plant of claim 1, wherein the propagation material is suitable for sexual reproduction, and a microspore, pollen, ovary, ovule, embryo sac or an egg cell, or the propagation material is suitable for vegetative reproduction and comprises a cutting, root, stem cell, or a protoplast, or the propagation material is suitable for tissue culture of regenerable cells or protoplasts and comprises a leaf, pollen, embryo, cotyledon, hypocotyl, meristematic cell, root, root tip, anther, flower or a stem, and wherein the propagation material comprises the modified lncRNA gene.

16. A method of producing a Cucumis melo plant comprising a modified lncRNA gene, comprising introducing the modified lncRNA gene in to the genome of the Cucumis melo plant, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
wherein the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1;
wherein the presence of the modified lncRNA gene confers improved shelf life of a fruit of the Cucumis melo plant as compared to fruit of an isogenic Cucumis melo plant lacking the modified lncRNA gene.

17. A method of selecting a Cucumis melo plant comprising a modified lncRNA gene, comprising:
a) identifying by genetic analysis the presence of the modified lncRNA gene in the genome of the Cucumis melo plant,
b) selecting a plant that comprises the modified lncRNA gene;
c) optionally further testing a fruit of the selected plant for improved shelf life as compared to fruit an isogenic Cucumis melo plant lacking the modified lncRNA gene, and
d) optionally selecting the further tested plant whose fruit exhibits improved shelf life as compared to fruit of an isogenic Cucumis melo plant lacking the modified lncRNA gene;
wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

18. The method of claim 17 further comprising the steps of:
c) further testing a fruit of the selected plant for improved shelf life as compared to fruit an isogenic Cucumis melo plant lacking the modified lncRNA gene, and
d) selecting the further tested plant whose fruit exhibits improved shelf life as compared to fruit of an isogenic Cucumis melo plant lacking the modified lncRNA gene.

19. A method for producing a Cucumis melo plant, comprising the steps of:
(a) crossing a first Cucumis melo parent plant whose genome comprises a modified lncRNA gene with a second Cucumis melo parent plant to obtain an F1 population;
(b) optionally performing one or more rounds of selfing and/or crossing with a F1 Cucumis melo plant to obtain a further generation population;
(c) selecting from the population resulting from the cross of step a) or from the further generation population of step b), a Cucumis melo plant that comprises the modified lncRNA gene;
wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

20. The method of claim 19, wherein the plant is grown from a seed deposited under NCIMB accession number 43973, or a progeny plant thereof that has retained the modified lncRNA gene.

21. A method for the production of hybrid Cucumis melo seed comprising: crossing a first Cucumis melo parent plant with a second Cucumis melo parent plant, and harvesting the hybrid seed;
wherein the first Cucumis melo parent plant or the second Cucumis melo parent plant comprises a modified lncRNA gene, wherein:
the wild type lncRNA gene has a coding sequence according to SEQ ID NO: 1, wherein the modified lncRNA gene encodes a modified lncRNA transcript, wherein the modified lncRNA transcript encoded by the modified lncRNA gene has a changed function, a reduced function, or it is non-functional as a result of the modification;
the modified lncRNA gene comprises a mutation, said mutation comprising a G1571A mutation at position 1571 of SEQ ID NO: 1, or at a corresponding position of a homologous sequence having at least 95% sequence identity to SEQ ID NO: 1; and
the presence of the modified lncRNA gene confers improved shelf life of a fruit of the Cucumis melo plant as compared to fruit of an isogenic Cucumis melo plant lacking the modified lncRNA gene.

22. The hybrid Cucumis melo seed produced by the method of claim 21.

23. The method of claim 21, wherein the second Cucumis melo parent plant comprises the modified lncRNA gene.

24-26. (canceled)
